(19) United States
(12) Patent Application Publication
(10) Pub. No.: US 2006/0293752 A1
(43) Pub. Date: Dec. 28, 2006

(54) Intervertebral disc prosthesis and associated methods

(76) Inventors: Missoum Moumene, Newton, MA (US); Martin Masson, Raynham, MA (US)

Correspondence Address: Maginot, Moore & Beck LLP, Chase Tower, Suite 3250, 111 Monument Circle, Indianapolis, IN, 46204-5109, US

(21) Appl. No.: 11/167,709

(22) Filed: Jun. 27, 2005

(51) Int. Cl. A61F 2/44

(52) U.S. Cl. 623/17.13; 623/17.14

(57) ABSTRACT

An intervertebral disc prosthesis comprises a first plate designed for attachment to an upper vertebra and a second plate designed for attachment to a lower vertebra. A prosthesis core is positioned between the first plate and the second plate. At least one spring is connected between the first plate and the second plate. The spring comprises a U-shaped torsion bar including a first leg and a second leg. The first leg of the U-shaped torsion spring is attached to the first plate and the second leg is attached to the second plate. The spring provides a selected amount of resistance to movement of the first plate relative to the second plate. In one embodiment, the selected resistance is provided by the attachment locations of the spring to the plates. In another embodiment, the selected resistance is provided by a predetermined spring rate of the springs of the prosthesis.

### BACKGROUND

This invention relates to the field of prosthetics, and more particularly, to an intervertebral disc prosthesis that is implantable to replace a damaged natural intervertebral disc and associated methods.

The human spine consists of twenty-four small bones known as vertebrae that protect the spinal cord and provide stability to the torso. The vertebrae are arranged in a column and stacked vertically upon each other. Between adjacent vertebra is a fibrous bundle of tissue called an intervertebral disc. These intervertebral discs act as a cushion to the spinal column by absorbing the shock and pressure associated with everyday movement. They also prevent the vertebrae from rubbing against each other.

Each intervertebral disc consists of two distinct regions. The firm outer region, anulus fibrosus, maintains the shape of the intervertebral disc. The inner region, nucleus pulposus, is comprised of a soft spongy tissue that enables the disc to function as a shock absorber. Over time, the normal aging process causes the intervertebral discs to degenerate, diminishing their water content and thereby reducing their ability to properly absorb the impact associated with typical movements of the body. Diminished water content in the intervertebral discs may also cause the vertebrae to move closer together. Tears and scar tissue can weaken the discs, resulting in injury. When the discs wear out or are otherwise injured, they can not function normally and may cause pain and limit activity. This condition is known as degenerative disc disease.

Degenerative disc disease can potentially be relieved by a surgical procedure called artificial disc replacement. In this procedure, the damaged natural intervertebral disc is replaced by a prosthetic disc. One existing design of an intervertebral prosthetic disc is disclosed in U.S. Pat. No. 5,556,431 issued to Büttner-Janz. The disc prosthesis disclosed in this patent is comprised of two metal endplates and a center polyethylene core. The center core includes an upper spherical surface portion and a lower spherical surface portion. The upper endplate includes a concave surface that fits upon and is congruent with the upper spherical surface of the core. The lower endplate includes a concave surface that fits under and is congruent with the lower spherical surface of the core. Another example of an existing design of an intervertebral prosthetic disc is disclosed in U.S. Pat. No. 5,401,269 issued to Büttner-Janz et al. During artificial disc replacement surgery, the damaged disc is first removed and the end surfaces of the exposed vertebrae are cleared of debris. The vertebrae are spread apart and the metal endplates are positioned on the respective vertebra and tapped into place. The polyethylene core is then inserted between the endplates and the vertebrae are returned to their normal position. The pressure of the spinal column further seats the endplates into the vertebral bones and secures the core in place.

One common challenge when designing intervertebral disc prosthesis of the type discussed above is to provide for stabilization of the disc prosthesis. In particular, it is desirable to limit the range of movement between the upper and lower endplates of the disc prosthesis in three dimensions, including the frontal plane (lateral bending), the sagittal plane (flexion), and the transversal plane (torsion). These three planes are shown diagrammatically in FIG. 21. When the range of movement between the upper and lower endplates is not limited, excessive loads may be transferred to the facets of adjoining vertebrae which is not desirable.

One manner of limiting the range of motion between the upper and lower endplates in an intervertebral disc prosthesis of the type identified above involves providing structural features on the upper and lower endplates that cooperate respectively with features on the center core to limit the extent of relative movement therebetween. One such structural arrangement involves the provision of an edge rim or radial collar around the core between its upper and lower spherical surfaces. U.S. Pat. No. 5,556,431 discloses this type of configuration. While such arrangement restricts movement of the endplates in the frontal and sagittal bending planes, such a collar does not restrict movement of the upper and lower endplates about the torsional axis. Thus, additional structural features have been introduced for restricting movement of the upper and lower endplates about the torsional axis. For example, one mechanism for restricting movement about the torsional axis involves the use of additional co-acting upper and lower structure provided at the upper and lower spherical surfaces of the core of the intervertebral disc prosthesis. Such a design is shown in U.S. Pat. No. 5,401,269.

Although the above described arrangements allow for restricted relative movement of the upper and lower plates in various planes, different types of movement restrictions may be desired. For example, it may be desirable to provide an intervertebral disc prosthesis that requires a relatively higher amount of force to move the end plates in relation to each other as one end plate closely approaches the restrictive collar of the core as compared to the amount of force required when the such end plate is spaced substantially apart from the restrictive collar. Such an arrangement would more closely resemble the functioning of a natural intervertebral disc. Furthermore, this type of arrangement would reduce wear of the core collar by reducing the frequency of end plate-to-collar contact.

In addition, this type of arrangement that limits relative movement between the upper and lower plate does not provide customized movement restriction depending upon the physical condition or circumstances of the particular patient. For example, the condition of a certain patient's spine may indicate that rotation in the transverse plane should be severely restricted, while allowing substantially more relative movement between the plates in the frontal and sagittal planes. As another example, very limited relative movement between the plates may be desired in all planes. Accordingly, it would be desirable to provide an intervertebral disc prosthesis that allows a surgeon to introduce varying resistances in different movement planes of the intervertebral disc, depending upon the needs or physical condition of the patient. This feature would enable a surgeon to better respond to the needs of its patients by individually customizing the intervertebral disc prosthesis' flexional, torsional, and lateral stability based on the degree of instability demonstrated at the time of implantation of the disc prosthesis in the patient.

### SUMMARY

An intervertebral disc prosthesis comprises a prosthesis core sandwiched between two endplates. The two endplates comprise a first plate including an outer perimeter edge and a second plate including an outer perimeter edge. The outer perimeter edge of the first plate and the outer perimeter edge of the second plate define an interior space of the disc prosthesis. The prosthesis core is positioned between the first plate and the second plate, with the endplates contacting the surface of the core. At least one torsion spring is connected between the first plate and the second plate.

The at least one torsion spring may comprise a U-shaped torsion bar. The U-shaped torsion bar includes a first leg and a second leg. The first leg of the U-shaped torsion bar is attached to the first plate and the second leg is attached to the second plate. The U-shaped torsion bar may be made of a number of different cross-sectional shapes, including round, oval, or square. In addition, the torsion spring itself may be formed in any one of a number of different shapes. For example, in one embodiment, the at least one torsion spring comprises two opposing U-shaped torsion bars. In another embodiment the at least one torsion spring comprises two facing U-shaped torsion bars. In yet another embodiment, the at least one torsion spring comprises a torsion bar forming an elliptical loop.

The at least one torsion spring provides resistance to movement of the first plate relative to the second plate. In one embodiment, the resistance provided by the at least one torsion spring is dependent upon a selected attachment location for the first leg on the fist plate and a selected attachment location for the second leg on the second plate. In particular, the attachment location for the first leg and the attachment location for the second leg determine the effective spring length for the at least one torsion spring, and this spring length determines the magnitude of resistance provided by the torsion spring. In another embodiment, the at least one torsion spring possesses a predetermined spring rate in order to provide a desired resistance. The spring rate may depend on features other than attachment location, such as cross-sectional area and cross-sectional shape. To aid a surgeon in identification of springs having differing spring rates, the springs are color coded based on their spring rates. Note that “spring rate” is defined as the rate of deflection in a particular direction versus amount of load applied, in other words, how much force is needed to bend a spring a given distance or twist a spring a given angle.

When the prosthesis is positioned within the body of a patient, the first plate is attached to the upper vertebra and the second plate is attached to the lower vertebra. The first plate includes a first plurality of teeth extending away from the core to assist with attachment of the first plate to the upper vertebra. Likewise, the second plate includes a second plurality of teeth extending away from the core to assist with attachment of the second plate to the lower vertebra. With the endplates positioned against the vertebrae, the core is positioned between the first plate and the second plate. At that time, the surgeon may determine that movement of the first plate relative to the second plate in one or more planes needs to be further restricted. If so, the physician then selects one of a plurality of different rated torsion springs operable to provide the desired movement resistance. Next, the surgeon attaches the first leg of the selected spring to the first plate and the second leg of the selected spring to the second plate. In one embodiment, the desired amount of resistance is provided to the intervertebral disc prosthesis by attaching the spring to first plate and the second plate at one of a plurality of attachment locations on each of the first and second plates. In another embodiment, the desired amount of resistance provided to the intervertebral disc prosthesis using one of a plurality of different springs having different spring rates or constants.

### BRIEF DESCRIPTION OF THE DRAWINGS

FIG. 1 shows an upper perspective view of an intervertebral disc prosthesis that incorporates the features of the present invention therein, with the disc prosthesis including a plurality of springs attached to the upper plate and the lower plate;

FIG. 2 shows a front cross-sectional view of the intervertebral disc prosthesis of FIG. 1 with the springs of the prosthesis removed for clarity of description;

FIG. 3 shows the front cross-sectional view of the intervertebral disc prosthesis of FIG. 2 with the upper plate rotated relative to its position shown in FIG. 2 to depict lateral bending;

FIG. 4 shows a side cross-sectional view of the intervertebral disc prosthesis of FIG. 1 with the springs of the prosthesis removed for clarity of description;

FIG. 5 shows the side cross-sectional view of the intervertebral disc prosthesis of FIG. 4 with the upper plate rotated relative to its position shown in FIG. 4 to depict flexion;

FIG. 6 shows the intervertebral disc prosthesis of FIG. 1, but showing the upper and lower plates modified and another plurality of springs substituted for the springs shown in FIG. 1;

FIG. 7 shows the intervertebral disc prosthesis of FIG. 1, but showing the upper and lower plates modified and yet another plurality of springs substituted for the springs shown in FIG. 1;

FIG. 8 shows the intervertebral disc prosthesis of FIG. 7, but showing the plurality of springs modified so as to possess an oval cross-sectional shape;

FIG. 9 shows the intervertebral disc prosthesis of FIG. 8, but showing the plurality of springs modified so as to possess a smaller cross-sectional shape at the U-shaped bends in the springs;

FIG. 10 shows the intervertebral disc prosthesis of FIG. 8, but showing the plurality of springs modified so as to possess a different oval cross-sectional shape in relation to that shown in FIG. 8;

FIG. 11 shows the intervertebral disc prosthesis of FIG. 1, but showing the upper and lower plates modified and another plurality of springs substituted for the springs shown in FIG. 1;

FIG. 12 shows the intervertebral disc prosthesis of FIG. 1, but showing the plurality of springs modified so as to possess an oval cross-sectional shape;

FIG. 13 shows the intervertebral disc prosthesis of FIG. 12, but showing the plurality of springs modified so as to possess a different oval cross-sectional shape in relation to that shown in FIG. 12;

FIG. 14 shows the intervertebral disc prosthesis of FIG. 1, but showing the upper and lower plates modified to provide differing spring attachment locations and another plurality of springs substituted for the springs shown in FIG. 1;

FIG. 15 shows the intervertebral disc prosthesis of FIG. 14, but showing another spring mounting arrangement having increased torsional stiffness substituted for the spring mounting arrangement shown in FIG. 14;

FIG. 16 shows the intervertebral disc prosthesis of FIG. 14, but showing yet another spring mounting arrangement having further increased torsional stiffness substituted for the spring mounting arrangement shown in FIG. 14;

FIG. 17 shows the intervertebral disc prosthesis of FIG. 1, but showing the upper and lower plates modified and another plurality of springs substituted for the springs shown in FIG. 1;

FIG. 18 shows the intervertebral disc prosthesis of FIG. 17, but showing the upper and lower plates as well as the spring mounting arrangement modified in relation to that shown in FIG. 17;

FIG. 19 shows the intervertebral disc prosthesis of FIG. 1, but showing the upper and lower plates modified and another plurality of springs substituted for the springs shown in FIG. 1;

FIG. 20A shows a top elevational view of the intervertebral disc prosthesis of FIG. 19;

FIG. 20B shows a side elevational view of the intervertebral disc prosthesis of FIG. 19;

FIG. 20C shows a front elevational view of the intervertebral disc prosthesis of FIG. 19; and

FIG. 21 shows exemplary planes of movement for an intervertebral disc prosthesis relative to a spine of a human body.

### DESCRIPTION

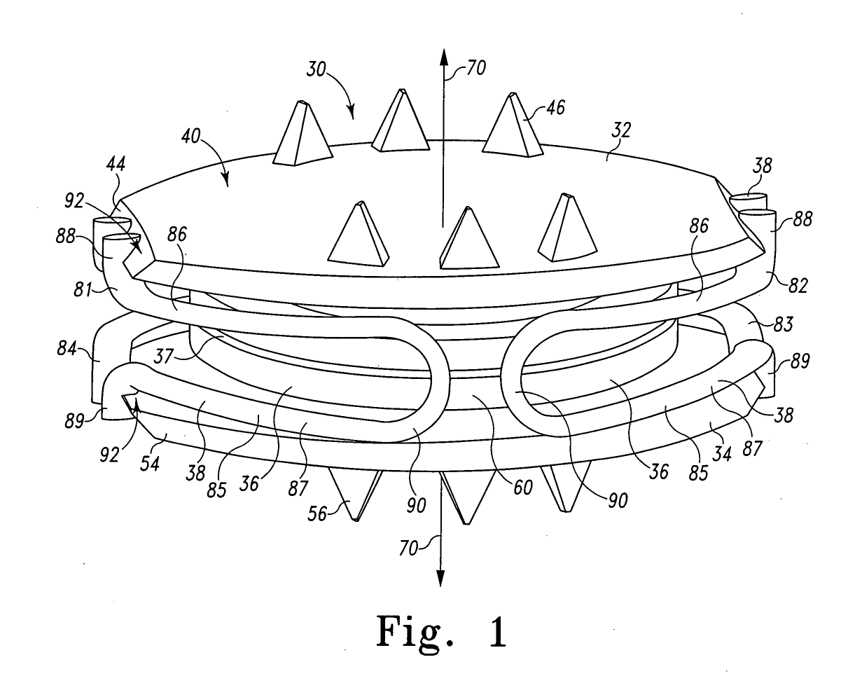

With reference to FIGS. 1-5, an intervertebral disc prosthesis 30 comprises an upper plate 32, a lower plate 34, a core 36, and a plurality of springs 38. The core 36 is sandwiched between the upper plate 32 and the lower plate 34. The upper plate 32 and the lower plate 34 ride upon the core 36 and are operable to move in relative to one another. The springs 38 are attached to the upper plate 32 and the lower plate 34 and provide resistance to movement of the upper plate relative to the lower plate. The springs are preferably made of a nitinol material. It should be appreciated that nitinol is a nonmagnetic alloy of titanium and nickel.

The upper plate 32 serves as a first endplate for the prosthetic device 30. The upper plate 32 is comprised of metal. In particular, the upper plate 32 is comprised of a medical grade cobalt chromium alloy. The upper plate 32 comprises an upper surface 40 on one side and a lower surface 42 on the other side. The upper plate 32 has an outer perimeter edge 44.

The upper surface 40 of the upper plate 32 is generally flat and is designed for engagement with a vertebral body. Teeth 46 are included on the upper surface 40 of the upper plate 32. The teeth 46 are designed to penetrate into the vertebral body, helping to secure the upper plate 32 to the vertebral body. Screws (not shown) may also be threaded through holes (not shown) in the upper plate to provide further assistance in securing the upper plate 32 to the vertebral body.

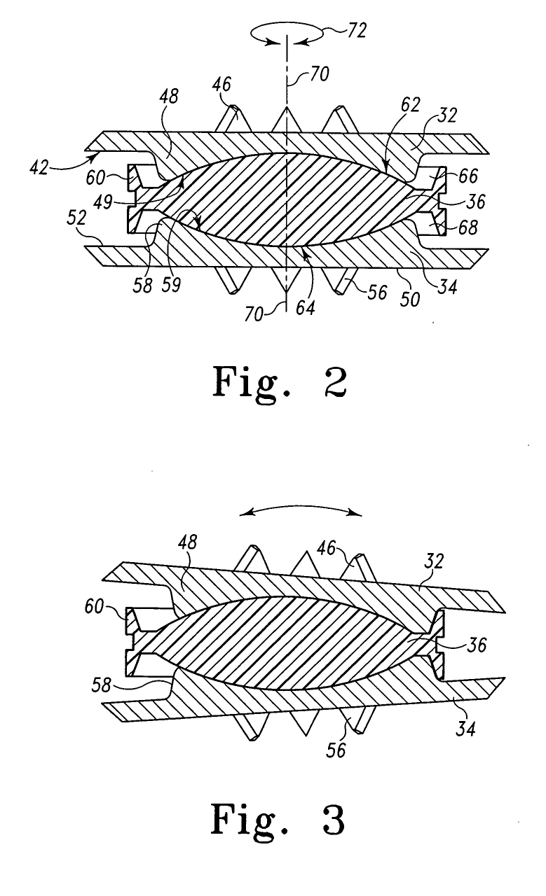

The lower surface 42 of the upper plate 32 is generally flat near the outer perimeter edge 44. As best seen in FIGS. 2-5, a neck portion 48 depends from the lower surface 42. An inner concave surface 49 is provided at the center of the neck portion 48.

The lower plate 34 is a mirror image of the upper plate 32 as shown in FIGS. 2-5, and is also made of a medical grade cobalt chromium alloy. The lower plate 34 includes a generally flat lower surface 50 outlined by an outer perimeter edge 54. A plurality of teeth 56 extend from the lower surface 50. The teeth 56 are designed to help secure the lower plate 34 to a vertebral body. The upper surface of the lower plate includes a neck portion 58 with an inner concave surface 59.

The prosthesis core 36 is sandwiched between the upper plate 32 and the lower plate 34. The core 36 is arranged within an interior space of the prosthesis 30 defined between the upper plate 32 and the lower plate 34. The prosthesis core 36 is comprised of a plastic material with good sliding (low friction) properties, such as ultra high molecular weight polyethylene. The prosthesis core 36 is generally disc shaped and possesses an outer radial collar 60, an upper spherical surface 62, and a lower spherical surface 64. As best seen in FIGS. 2-4, a first groove 66 is formed between the collar 60 and the upper spherical surface 62. A second groove 68 is formed between the collar 60 and the lower spherical surface 64. Also, a circumferential recess 37 is formed in the core 36 at its outer periphery as shown in FIG. 1. The recess 37 is configured to receive a radio opaque ring (not shown) which functions to identify location of the intervertebral disc prosthesis 30 during medical imaging.

When the prosthesis 30 is assembled, the concave surface 49 of the upper plate 32 and the upper spherical surface 62 of the core 36 form articular surfaces that slidingly contact each other. Likewise, the concave surface 59 of the lower plate 34 and the lower spherical surface 64 of the core 36 form articular surfaces that slidingly contact each other.

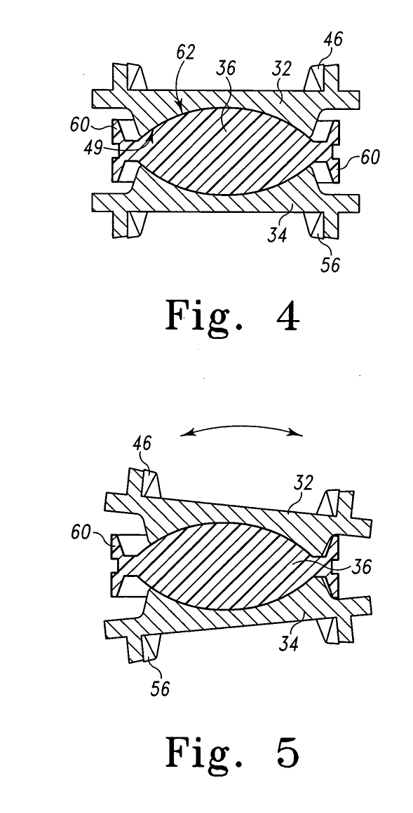

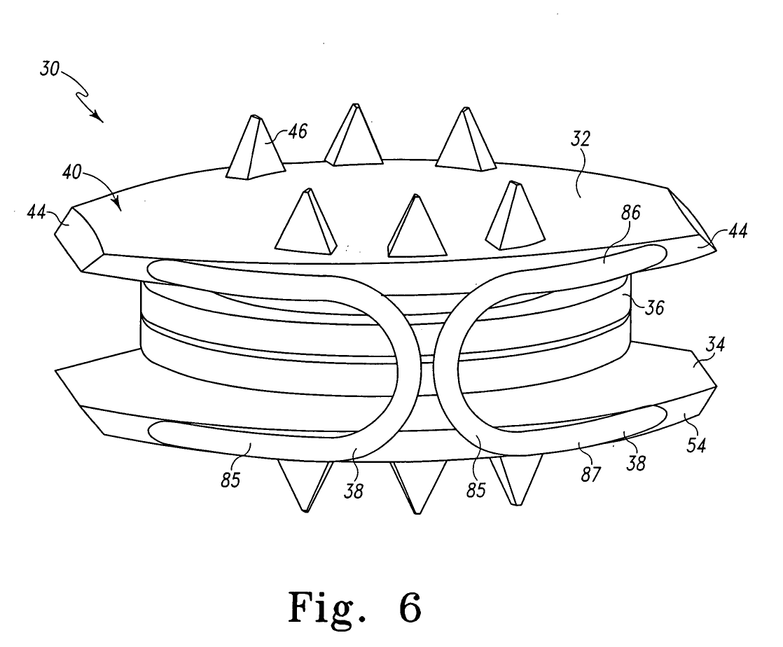

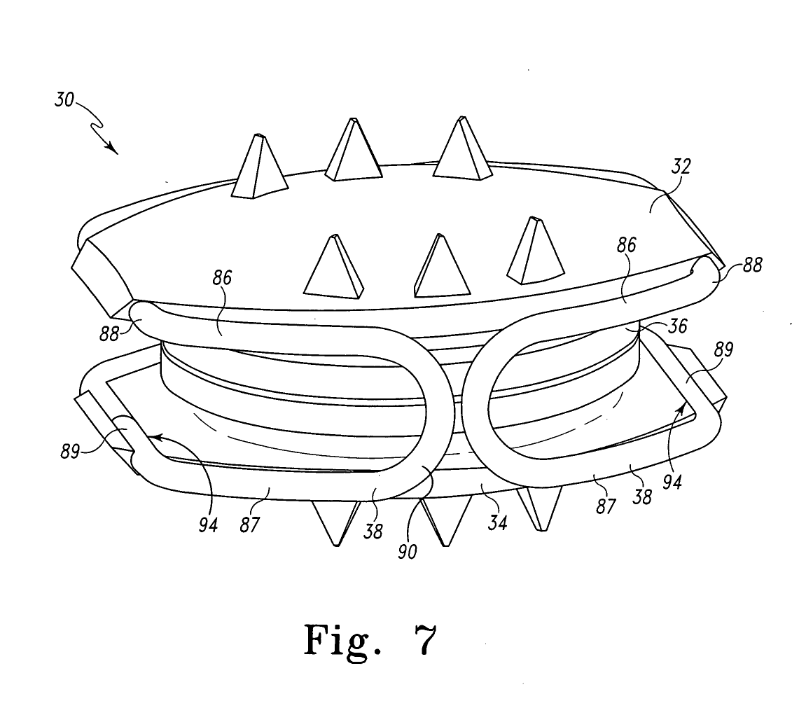

In the embodiment as shown in FIGS. 2-5, the articular surfaces 49, 62, 59, 64 are not perfectly spherical, but more closely resemble an ellipsoid. In particular, the radii of curvature are smaller in the sagittal section (see FIGS. 4 and 5) than in the frontal section (see FIGS. 2 and 3). Accordingly, when the end plates 32 and 34 are rotated in relation to the core 36 around the vertical axis 70 in the transversal plane (in the direction of arrow 72 in FIG. 2) the articular surface congruence is no longer present. This torsional movement results in the prosthesis plates being forced in relation to each other against the load of the body weight on them. As a consequence, the body weight causes a counteracting force which attempts to rotate the prosthesis plates back into the position in which the articular surfaces 49, 62, 59, 64 are in congruence. Thus, the configuration of the articulating surfaces 49, 62, 59, 64 may be used to provide a resistance to torsional movement of the endplates 32, 34.

In another embodiment (not shown in the drawings), the articular surfaces 49, 62, 59, 64 are completely spherical and remain congruous during torsional rotation of the end plates 32, 34 in relation to the core 36 around the vertical axis 70. In this embodiment, the radii of the arcs in the frontal (lateral bending) planes are equal to the radii of the arcs in the sagittal (flexion) planes. This allows the plates 32 and 34 to rotate upon the core 36, including rotation in the transversal plane (torsion) while the articular surfaces remain in congruous contact. In this embodiment, the articular surfaces 49, 62, 59, 64 do not offer significant resistance to torsional rotation.

With reference to FIGS. 2, 3 and 5, the radial collar 60 and associated grooves 66 and 68 provide for limited movement of the endplates in relation to the core 36 in the frontal (lateral bending) plane and sagittal (flexion) plane. In particular, at a certain angle of rotation of the upper plate 32 relative to the lower plate 34 in the frontal and sagittal planes, the collar 60 of the prosthesis core engages the neck portions 48 and 58 of the endplates 32, 34. This provides a defined stop against excessive rotation in the frontal (lateral bending) plane and sagittal (flexion) plane of the prosthesis 30, and effectively prevents the core from being expulsed form its location between the endplates.

With reference to FIG. 1, the plurality of springs 38 connect the upper plate 32 to the lower plate 34. The plurality of springs do not contact the core 36. In FIG. 1, the plurality of springs 38 include a first torsion spring 81, a second torsion spring 82, a third torsion spring 83 and a fourth torsion spring 84. The torsion springs are generally comprised of a resilient metallic material. In one embodiment, the springs are comprised of titanium. In another embodiment, the springs are comprised of stainless steel. However, the springs may be comprised of any biocompatible material with adequate spring properties, such as certain plastics or a composite of carbon and plastic.

Each torsion spring 81-84 comprises a generally U-shaped torsion bar 85 including a first leg 86, a second leg 87 and a U-shaped turn portion 90. The first leg 86 of the U-shaped torsion bar 85 is attached to the upper plate 32. The second leg 87 of the U-shaped torsion bar 85 is attached to the lower plate 34.

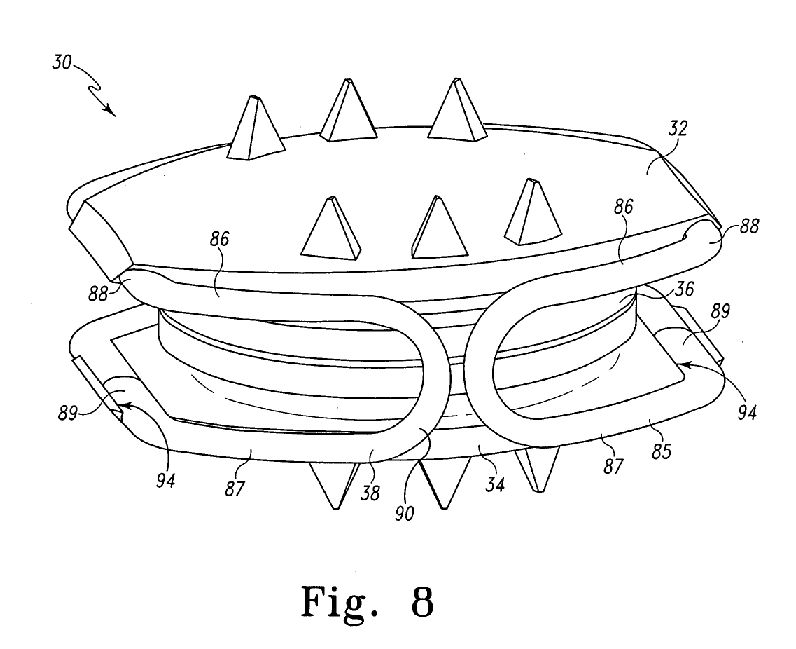

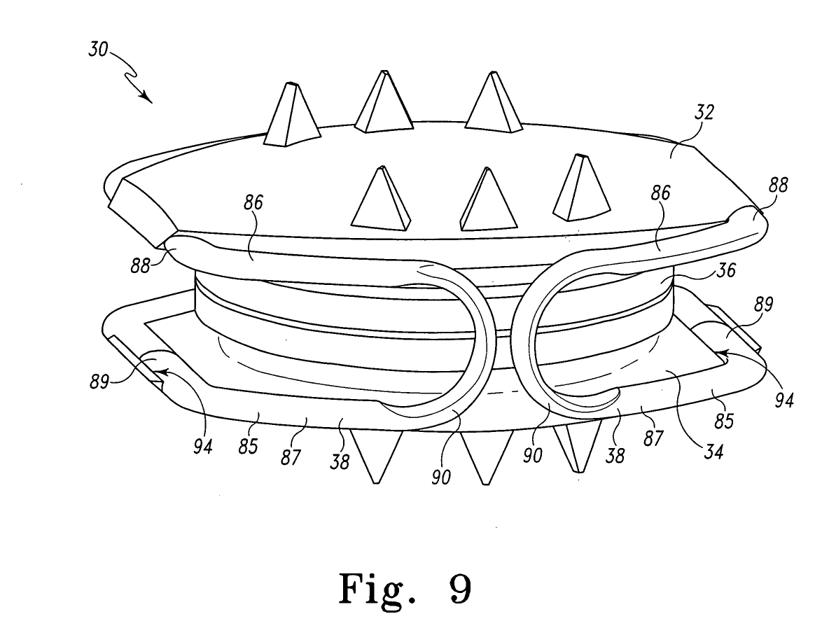

The legs 86, 87 of the U-shaped torsion bar 85 may be attached to the upper plate 32 and the lower plate 34 in any one of a number of different manners. As shown in FIG. 1, each leg 86, 87 includes an attachment foot 88, 89. Each foot 88, 89 is inserted in one of a plurality of small cut-out portions or cavities 92 formed in the upper plate 32 and the lower plate 34. The small cut-out portions 92 are designed to receive the feet 88, 89. The feet 88, 89 are retained within the cut-out portions 92 using a clip-on arrangement. In particular, the openings to the cut-out portions 92 are slightly smaller than the diameter of the feet 88, 89 such that the feet must be forced through the openings in a friction fit manner. Alternatively other materials or methods may be used to fix the feet 88, 89 to the cut-out portions 92, such as adhesives, solder, welding, or screws. For example, in the embodiment of FIG. 6, the ends of the legs 86, 87 are inserted into holes formed in the perimeter 44, 54 of the upper plate 32 and the lower plate 34 and an adhesive is used to retain the legs in the holes. In the embodiments shown in FIGS. 7-10, channels 94 are formed across the lower surface 42 of the upper plate 32 and opposing channels 94 are formed across the upper surface 52 of the lower plate 34. The feet 88, 89 are snapped in place in the channels after an adhesive material is placed in the channels. Other exemplary methods for attaching the springs 38 to the plates are also described below.

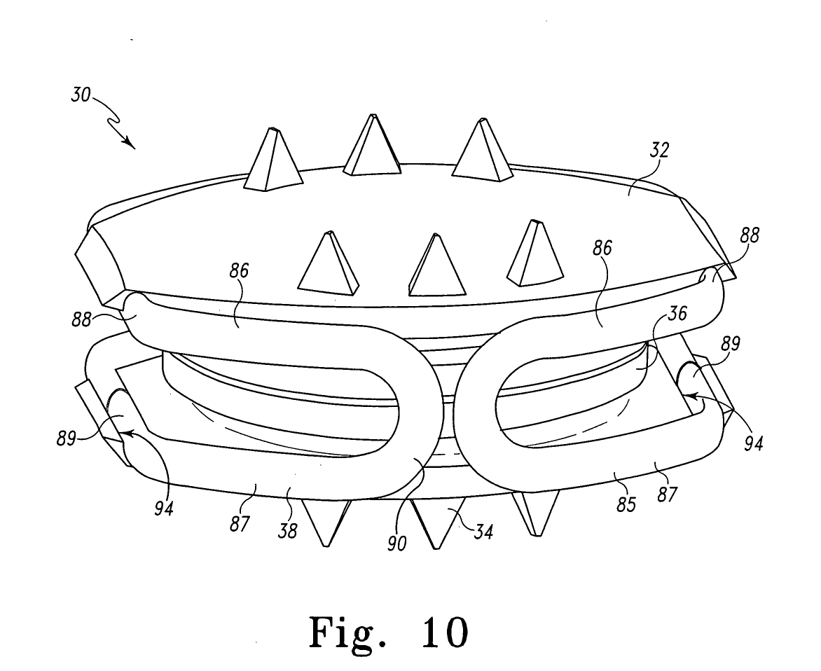

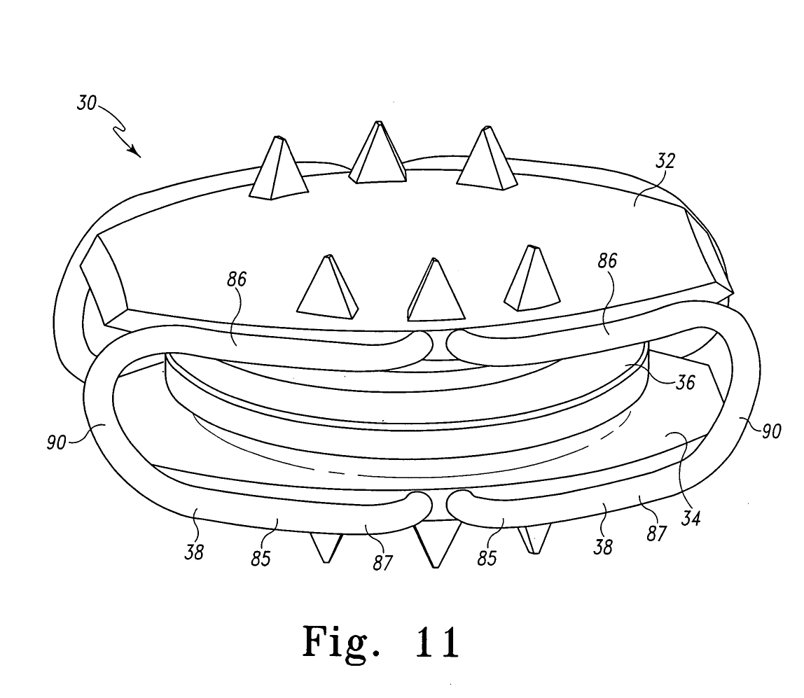

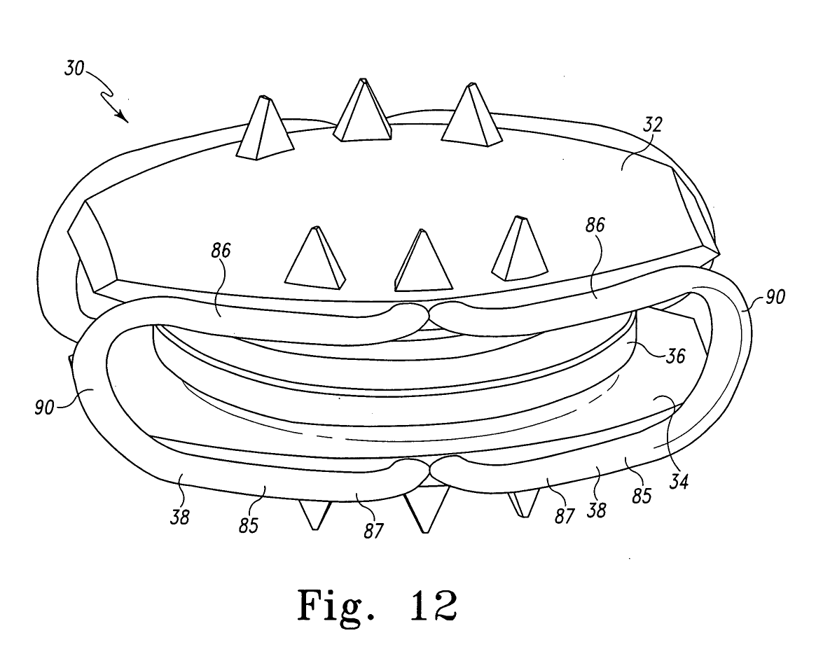

The U-shaped torsion bar 85 may be configured to possess any one of a number of different cross-sectional shapes, including round, elliptical, or square. The different cross-sectional shapes available allow different torsion bars 85 to offer different amounts of resistance in different rotational planes. For example FIG. 6 shows an intervertebral disc prosthesis 30 with springs 38 of round cross-sectional shape. FIG. 7 shows an intervertebral disc prosthesis with springs of elliptical cross-sectional shape. FIG. 8 shows an intervertebral disc prosthesis with springs of elliptical cross-sectional shape, wherein the cross-sectional ellipse is positioned ninety degrees from the cross-sectional ellipse of FIG. 7. FIG. 9 shows an intervertebral disc prosthesis with springs of elliptical cross-sectional shape, wherein the cross-sectional area is significantly reduced near the U-shaped turns 90 in the springs 38. This reduced cross-sectional area allows the springs 38 to bend more easily near the U-shaped turn portions 90. FIG. 10 shows an intervertebral disc prosthesis with springs 38 of an elliptical cross-sectional shape, wherein the springs are significantly thicker than those shown in FIG. 7. This allows the springs of FIG. 10 to provide significantly more resistance to relative rotation of the endplates in all planes than the springs of FIG. 7. In another embodiment, this would allow the springs of FIG. 10 to provide the same resistance to relative rotation of the endplates in all planes in comparison to the springs of FIG. 7 if the material selected for the springs of FIG. 10 possessed a lower modulus of elasticity than that of the springs of FIG. 7.

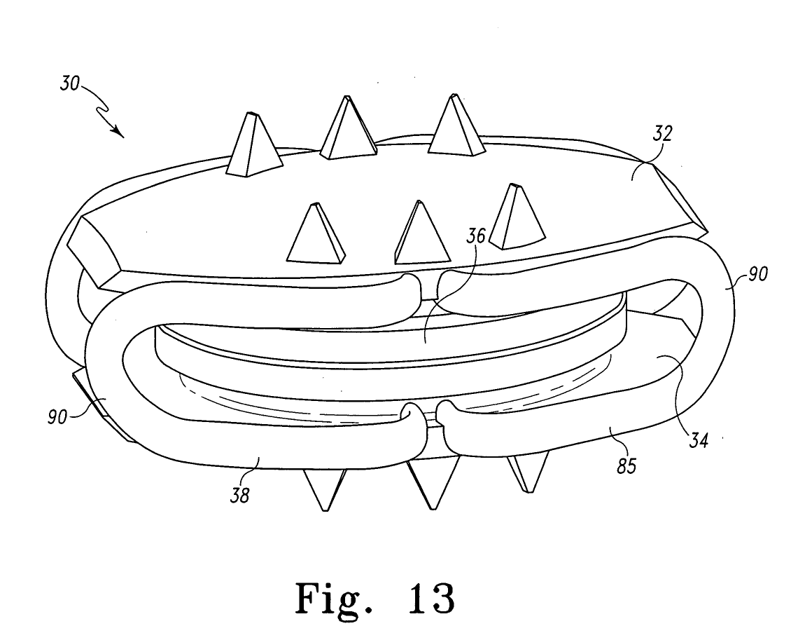

In addition to modifying the shape of the torsion springs, the arrangement of the torsion springs 38 may be modified to allow the springs to offer different resistances in different planes. For example, in the embodiment of FIG. 1, the torsion springs comprise two sets of opposing U-shaped torsion bars 85. In the embodiment of FIG. 6, only one set of opposing U-shaped torsion bars 85 is provided. As another example, FIG. 1 shows two opposing sets of U-shaped torsion bars 85, while FIG. 11 shows two facing sets of U-shaped torsion bars 85. These different arrangement options for the torsion springs allows the springs to be arranged in a manner that provides a desired amount of resistance in the desired planes, depending on the needs or physical condition of the patient.

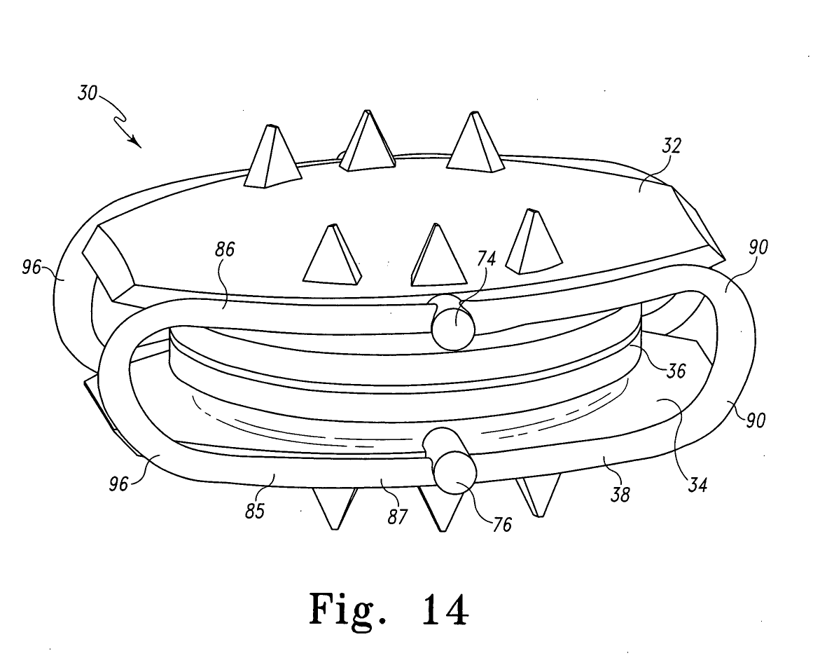

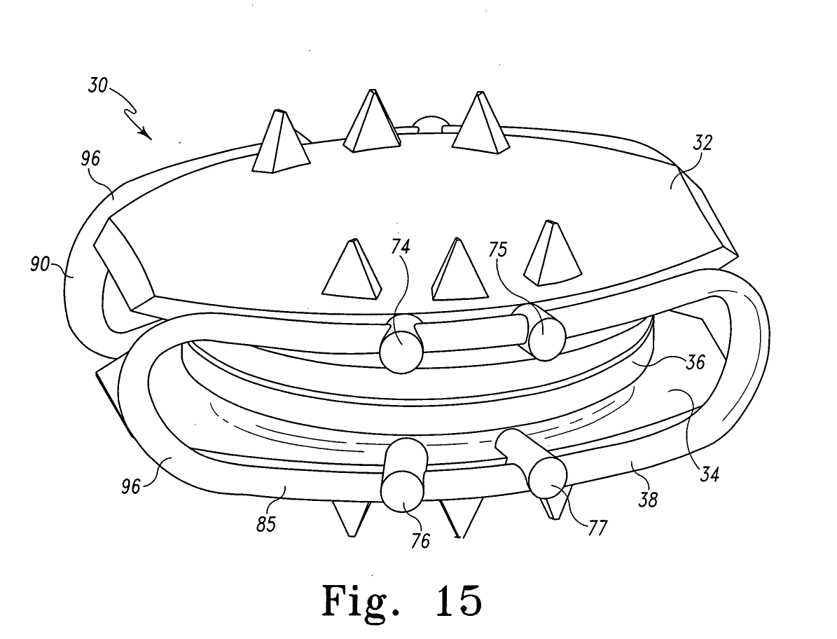

FIGS. 12 and 13 also show the arrangement of two sets of facing U-shaped torsion bars similar to that of FIG. 11. However, the cross-sectional shape of the torsion bars is different in FIGS. 12 and 13 from that of FIG. 11. Accordingly, it should be clear that numerous options exist for the shape and arrangement of the springs, and the intervertebral disc prosthesis may be designed with different numbers, sizes, shapes and arrangements of springs, depending upon the needs of the patient.

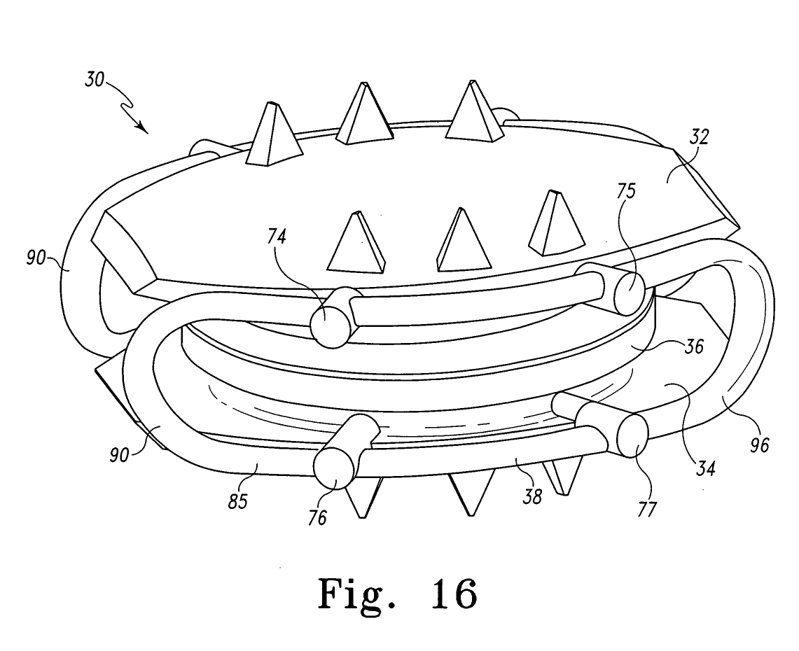

FIG. 14 shows an intervertebral disc prosthesis 30 wherein the springs 38 comprise two elliptical springs 96. One elliptical spring is mounted on an anterior side of the prosthesis 30 and another elliptical spring is mounted on a posterior side of the prosthesis. Each elliptical spring may be comprised of two U-shaped springs, or alternatively, be made of an integral, continuous, oval or collapsed (pre-stressed) circular ring. Each elliptical spring 96 is attached to the endplates 32, 34 with at least one upper mount 74 and at least one lower mount 76. The upper mount 74 and lower mount 76 are respectively secured to and extend from the outer perimeters of the upper plate 32 and lower plate 34. The mounts 74, 76 may be integral with the end plates 32, 34 or may be secured to the end plates in a similar manner as the springs described in the preceding embodiments, such as affixed to holes or grooves in the plates using adhesives, mechanical fasteners, press-fit, or snap-fit arrangements. The use of mounts 74, 76 to attach the springs to the endplates demonstrates that the springs may be either indirectly attached to the endplates or directly attached to the endplates as described above according to the principles of the present invention.

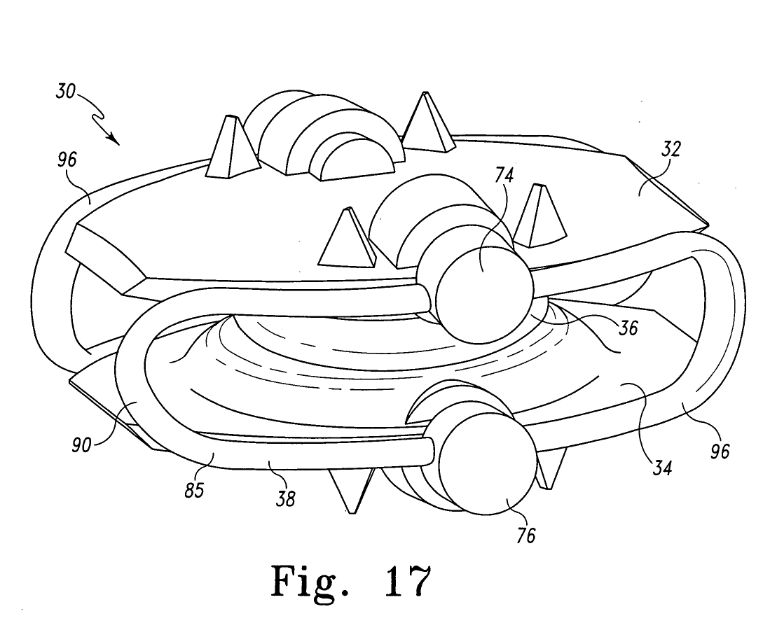

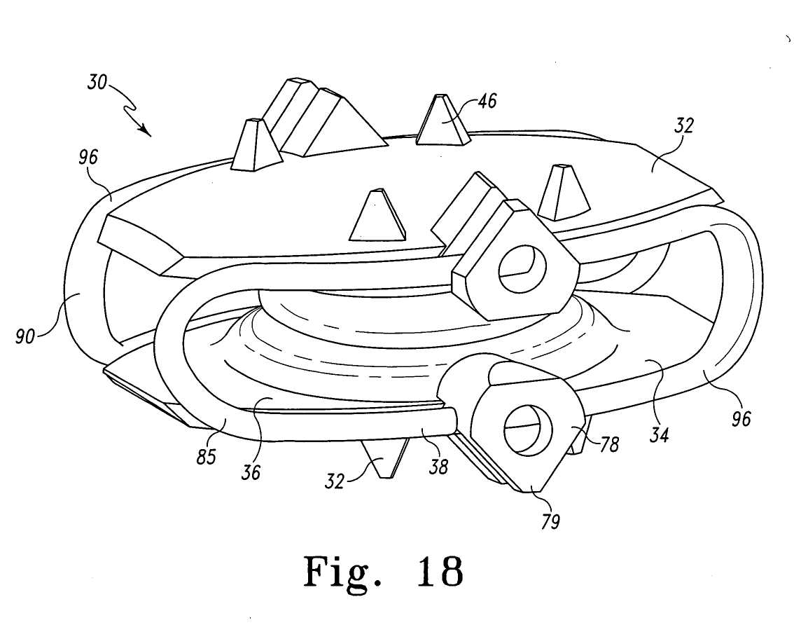

With continued reference to FIG. 14 as well as FIGS. 15 and 16, in one alternative embodiment of the disc prosthesis 30, the resistance provided by the torsion springs 38 is dependent upon a selected attachment location for the first leg to the upper plate and a selected attachment location for the second leg to the lower plate. In this embodiment, the attachment location for the first leg and the attachment location for the second leg determines the effective spring length for the at least one torsion spring, and this spring length determines the resistance provided by the torsion spring. For example, in FIG. 14, only one attachment location is provided for each spring per plate, as shown by mounts 74 and 76. Therefore a relatively long effective spring length is offered by the elliptical spring 96, resulting in a relatively low resistance being provided by the spring. However, in FIG. 15 two separate attachment locations are provided for each spring per plate, as shown by mounts 74, 75, 76 and 77. This results in a shorter effective spring length for the elliptical spring 96, resulting in a greater resistance being provided by the spring of FIG. 15 compared to that of FIG. 14 (assuming the spring rates are otherwise the same). In FIG. 16, the distance between the two attachment locations per plate is greater than that of FIG. 15. This results in an even shorter effective spring length and even greater resistance to movement of the plates provided by the spring.

As an example of the difference that attachment location can make to spring resistance, a spring arrangement similar to FIG. 14 with a single mount for each spring per plate offers the following resistances: 3.6 Nm resistance to torsion; 5.5 Nm resistance to flexion; and 2.4 Nm resistance to lateral bending. By contrast, a spring arrangement similar to FIG. 16 with two separated mounts for each spring per plate offers the following resistances: 7.5 Nm resistance to torsion; 14.1 Nm resistance to flexion; and 14.3 Nm resistance to lateral bending.

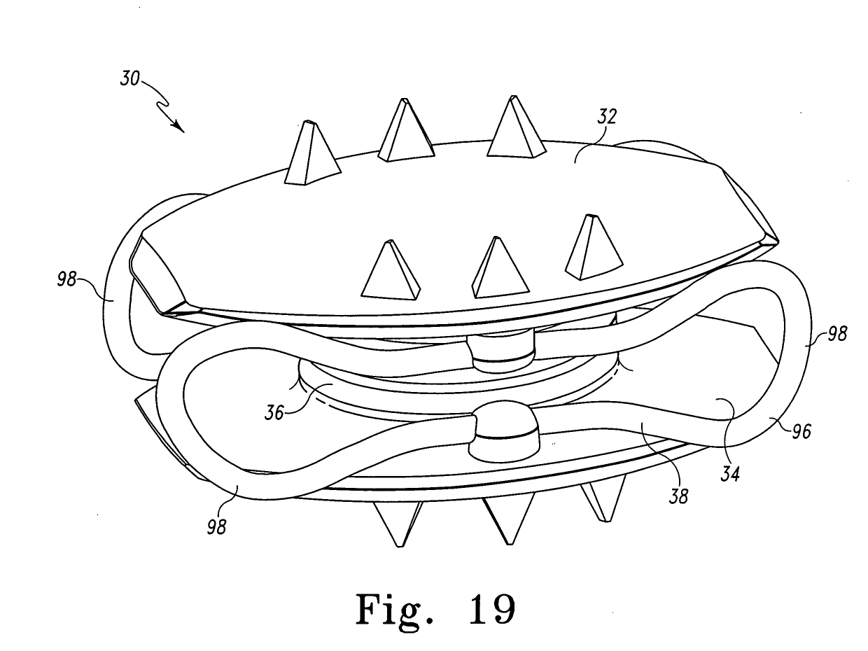

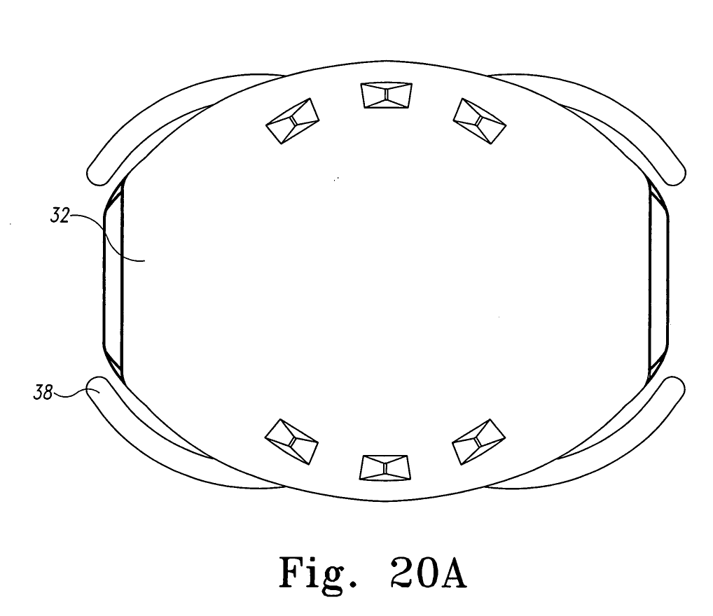

FIGS. 17 and 18 show that different types of mounts may be used to attach the springs 38 to the end plates. The mounts 78 shown in FIG. 17 are wider than the mounts 74, 76 of FIG. 14, and the size of the mounts 78 reduces the effective spring length of the spring 38. The wider mounts of FIG. 17 may also add stability to the connection between the mounts 78 and the plates 32, 34. In addition to being enlarged, the mounts 78 shown in FIG. 18 also include teeth 79, similar to the teeth 46 on the endplates 32, 34. These teeth are designed to seat into the facing vertebra and help secure the prosthesis 30 in place on the vertebra. The wider mounts of FIG. 18 have holes that may be tapered or threaded to receive corresponding taper mounts or threaded screws for affixing the springs to the endplates.

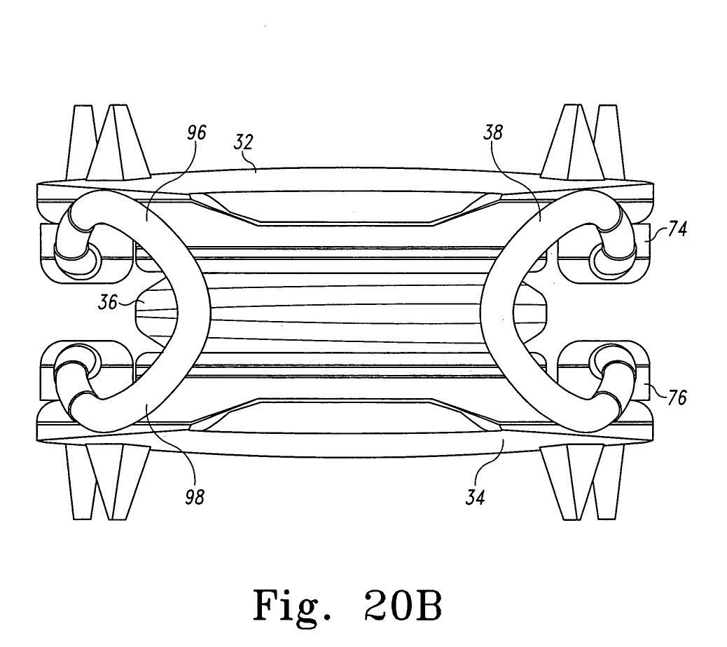

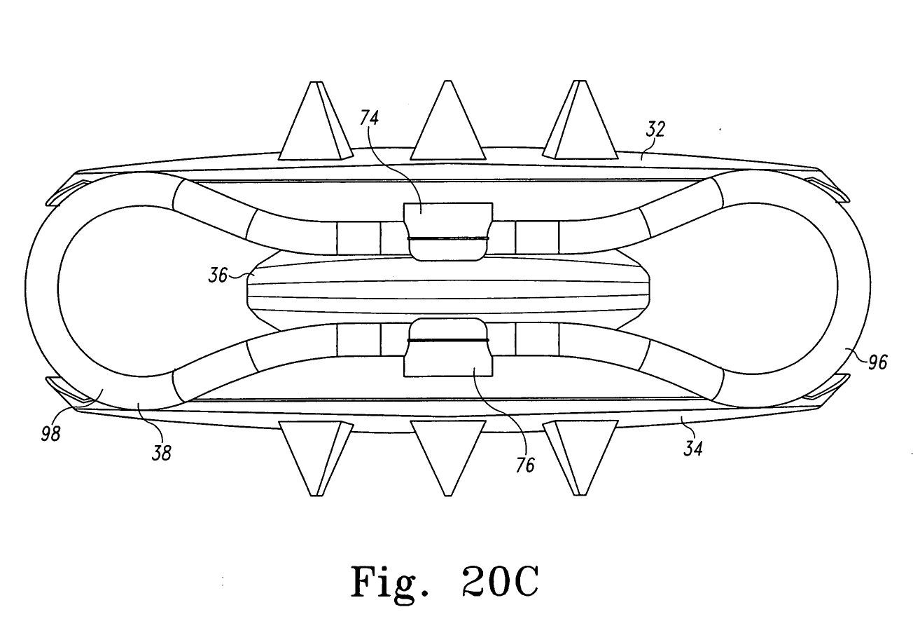

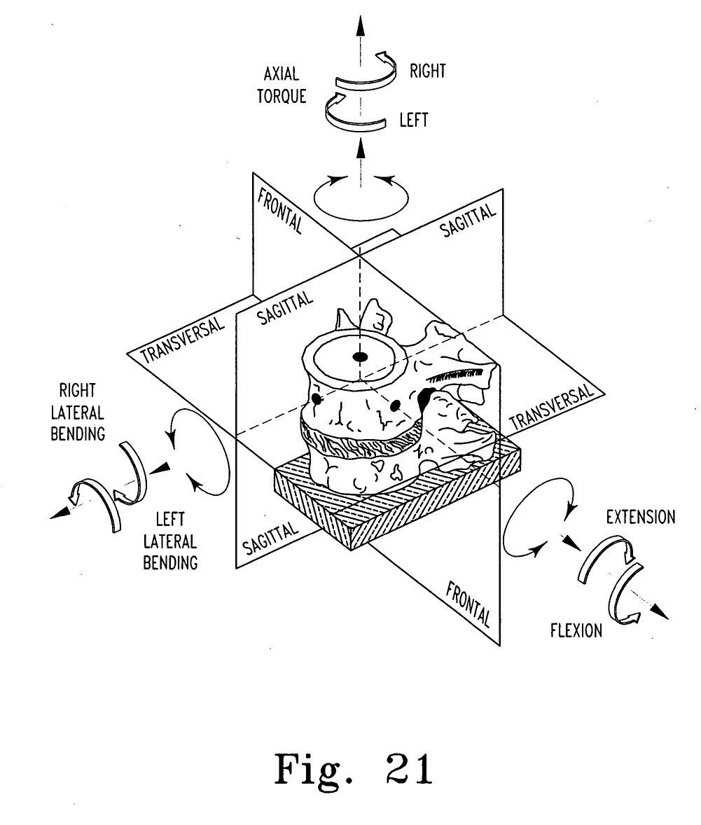

FIGS. 19-20C show yet another embodiment of an intervertebral disc prosthesis 30 with generally elliptical springs 96. In this embodiment, the elliptical springs 96 include flared U-shaped ends 98 having a wider diameter than the U-shaped ends shown in FIGS. 14-18. The flared U-shaped ends 98 generally increase the effective spring length and reduce the resistance provided by the spring. The upper and lower mounts 74, 76 shown in FIGS. 19-20C are clip-on type mounts that enable each spring to be secured to the mounts without the use of tools, fasteners or adhesives.

In yet another embodiment of the intervertebral disc prosthesis 30, different spring resistances may be selected by using springs with different spring rates. In this embodiment, if a greater resistance is desired, a spring with a greater spring rate is chosen. If a smaller resistance is desired, a spring is selected with a smaller spring rate. In this embodiment, a plurality of different springs with different spring rates are made available to the surgeon in a kit. The springs are color coded to indicate their respective spring rate. For example, a red spring may indicate a spring with a high spring rate. A yellow spring may indicate a spring with a medium level spring rate. A green spring may indicate a spring with a low spring rate. Of course, actual spring rates are determined by the shape of the spring, including cross-sectional area, as well as the material used to create the spring. With this arrangement, the surgeon may quickly choose a spring before or even during surgery to provide the desired amount of resistance to movement of the endplates relative to each other.

Before the intervertebral disc prosthesis 30 is implanted in a patient, the surgeon first selects a desired amount of resistance to movement of the upper plate 32 relative to the lower plate 34. The surgeon then selects the appropriate springs and/or attachment locations and/or spring arrangements that will provide the desired resistance to movement in each of the frontal, sagittal and transversal planes. After the appropriate springs, attachment locations, and spring mounting arrangements are selected, the spring or springs 38 for the posterior side of the prosthesis 30 are attached between the upper and lower plates 32, 34. The surgeon then uses an anterior approach to access and remove the patient's damaged disc. The end surfaces of the vertebrae that formerly sandwiched the damaged disc are then spread apart. Thereafter, the endplates 32, 34 of the intervertebral disc prosthesis 30 are positioned on the respective vertebrae and tapped into place. Next, the core 36 is inserted between the endplates 32, 34 and the surgeon affixes the second spring or spring set on the anterior side of the prosthesis 30. The vertebrae are then returned to their normal position. The pressure of the spinal column seats the endplates into the vertebrae and secures the prosthesis 30 in place. In an alternative embodiment with the surgeon also performing an anterior approach, the two springs 38 or sets of springs are attached laterally.

Although the present invention has been described with respect to certain preferred embodiments, it will be appreciated by those of skill in the art that other implementations and adaptations are possible. For example, although only U-shaped torsion springs have been discussed above, any number of differently shaped springs could be used with the intervertebral disc prosthesis according to the principles of the present invention. Indeed, S-shaped or Z-shaped springs could be used between the upper plate and the lower plate of the prosthesis. Additionally, although the present invention mainly describes torsional spring embodiments, it should be appreciated that any springs that oppose resistance to flexion mainly in the sagittal or mainly in the frontal planes may be devised and utilized. Further, as the resistance provided by the spring seldom occurs in a single plane, it should be appreciated that springs may be devised and utilized for the purpose of offering coupled resistance to movement, in other words, non-linear, coupled stiffness characteristics that mimic those associated with a natural disc. In another embodiment of the present invention, the disc prosthesis may be a pre-assembled, dynamically stabilized disc including endplates, core and springs, thereby eliminating the need for assembly at the time of surgery. As another example, the springs between the upper and lower plates reduces the need for the radial collar which acts as a definite stop, and thus the intervertebral disc prosthesis could be configured so as not to possess such a radial collar. If the radial collar were removed, the design of the disc prosthesis could be more compact. Thus, another embodiment of the present invention contemplates a prosthesis similar to the prosthesis shown in FIG. 1, except that the radial collar 60 of the core 36 is eliminated from the prosthesis. In addition, while the intervertebral disc prosthesis 30 was described above as being implanted by way of an anterior approach, the prosthesis 30 may be implanted by any other approach such as a posterior approach and a lateral approach. Further, while the intervertebral disc prosthesis disclosed herein is described as being used to replace a damaged natural intervertebral disc, it should be appreciated that it may also be used to replace a damaged prosthetic intervertebral disc. Moreover, there are advantages to individual advancements described herein that may be obtained without incorporating other aspects described above. Therefore, the spirit and scope of the appended claims should not be limited to the description of the preferred embodiments contained herein.

What is claimed is:

1. An intervertebral disc prosthesis, comprising:
an upper prosthesis component having (i) a first vertebra facing surface, (ii) a first bearing surface, and (iii) at least one first vertebra engagement tooth extending from said first vertebra facing surface;
a lower prosthesis component having (i) a second vertebra facing surface, (ii) a second bearing surface, and (iii) at least one second vertebra engagement tooth extending from said second vertebra facing surface;
an intermediate prosthesis component interposed between said upper prosthesis component and said lower prosthesis component, said intermediate prosthesis component having (i) a third bearing surface positioned in contact with said first bearing surface of said upper prosthesis component, and (ii) a fourth bearing surface positioned in contact with said second bearing surface of said lower prosthesis component; and
at least one spring coupled to both said upper prosthesis component and said lower prosthesis component.

2. The intervertebral disc prosthesis of claim 1, wherein said at least one spring is spaced apart from said intermediate prosthesis component.

3. The intervertebral disc prosthesis of claim 1, wherein:
said at least one spring is in a relaxed state when said upper prosthesis component is located at a first position in relation to said lower prosthesis component, and
said at least one spring is in a stressed state when said upper prosthesis component is located at a second position in relation to said lower prosthesis component.

4. The intervertebral disc prosthesis of claim 1, wherein said at least one spring includes:
a first end connected to said lower prosthesis component, and
a second end connected to said upper prosthesis component.

5. The intervertebral disc prosthesis of claim 4, wherein said at least one spring comprises a U-shaped member that includes (i) a first leg defining said first end, and (ii) a second leg defining said second end.

6. The intervertebral disc prosthesis of claim 1, wherein:
said first bearing surface of said upper prosthesis component slidingly contacts said third bearing surface of said intermediate prosthesis component during relative movement between said upper prosthesis component and said intermediate prosthesis component, and
said second bearing surface of said lower prosthesis component slidingly contacts said fourth bearing surface of said intermediate prosthesis component during relative movement between said lower prosthesis component and said intermediate prosthesis component.

7. The intervertebral disc prosthesis of claim 1, wherein said at least one spring is made of a nitinol material.

8. An intervertebral disc prosthesis, comprising:
an upper prosthesis component having (i) a first vertebra facing surface, and (ii) a first bearing surface;
a lower prosthesis component having (i) a second vertebra facing surface, and (ii) a second bearing surface;
an intermediate prosthesis component interposed between said upper prosthesis component and said lower prosthesis component, said intermediate prosthesis component having (i) a third bearing surface positioned in contact with said first bearing surface of said upper prosthesis component, and (ii) a fourth bearing surface positioned in contact with said second bearing surface of said lower prosthesis component; and
at least one spring coupled to both said upper prosthesis component and said lower prosthesis component.

9. The intervertebral disc prosthesis of claim 8, wherein said at least one spring is spaced apart from said intermediate prosthesis component.

10. The intervertebral disc prosthesis of claim 8, wherein:
said at least one spring is in a relaxed state when said upper prosthesis component is located at a first position in relation to said lower prosthesis component, and
said at least one spring is in a stressed state when said upper prosthesis component is located at a second position in relation to said lower prosthesis component.

11. The intervertebral disc prosthesis of claim 8, wherein said at least one spring includes:
a first end connected to said lower prosthesis component, and
a second end connected to said upper prosthesis component.

12. The intervertebral disc prosthesis of claim 11, wherein said at least one spring comprises a U-shaped member that includes (i) a first leg defining said first end, and (ii) a second leg defining said second end.

13. The intervertebral disc prosthesis of claim 8, wherein:
said first bearing surface of said upper prosthesis component slidingly contacts said third bearing surface of said intermediate prosthesis component during relative movement between said upper prosthesis component and said intermediate prosthesis component, and
said second bearing surface of said lower prosthesis component slidingly contacts said fourth bearing surface of said intermediate prosthesis component during relative movement between said lower prosthesis component and said intermediate prosthesis component.

14. The intervertebral disc prosthesis of claim 8, wherein said at least one spring is made of a nitinol material.

15. An intervertebral disc prosthesis, comprising:
an upper prosthesis component having (i) a first vertebra facing surface, and (ii) a first bearing surface;
a lower prosthesis component having (i) a second vertebra facing surface, and (ii) a second bearing surface;
an intermediate prosthesis component interposed between said upper prosthesis component and said lower prosthesis component, said intermediate prosthesis component having (i) a third bearing surface positioned in contact with said first bearing surface of said upper prosthesis component, and (ii) a fourth bearing surface positioned in contact with said second bearing surface of said lower prosthesis component;
a first spring coupled to both said upper prosthesis component and said lower prosthesis component, said first spring possessing a first spring rate; and
a second spring coupled to both said upper prosthesis component and said lower prosthesis component, said second spring possessing a second spring rate which is different from said first spring rate.

16. The intervertebral disc prosthesis of claim 15, wherein both first spring and said second spring are spaced apart from said intermediate prosthesis component.

17. The intervertebral disc prosthesis of claim 15, wherein:
each of said first spring and second spring is in a relaxed state when said upper prosthesis component is located at a first position in relation to said lower prosthesis component, and
each of said first spring and said second spring is in a stressed state when said upper prosthesis component is located at a second position in relation to said lower prosthesis component.

18. The intervertebral disc prosthesis of claim 15, wherein each of said first spring and said second spring includes:
a first end connected to said lower prosthesis component, and
a second end connected to said upper prosthesis component.

19. The intervertebral disc prosthesis of claim 18, wherein each of said first spring and said second spring comprises a U-shaped member that includes (i) a first leg defining said first end, and (ii) a second leg defining said second end.

20. The intervertebral disc prosthesis of claim 15, wherein:
said first bearing surface of said upper prosthesis component slidingly contacts said third bearing surface of said intermediate prosthesis component during relative movement between said upper prosthesis component and said intermediate prosthesis component, and
said second bearing surface of said lower prosthesis component slidingly contacts said fourth bearing surface of said intermediate prosthesis component during relative movement between said lower prosthesis component and said intermediate prosthesis component.

21. The intervertebral disc prosthesis of claim 15, wherein said at least one spring is made of a nitinol material.

22. A method, comprising:
determining a desired amount of relative movement restriction between a first vertebra and a second vertebra of a patient based on physical condition of the patient;
providing a kit having a plurality of springs including (i) a first spring possessing a first spring rate, and (ii) a second spring possessing a second spring rate that is different from said first spring rate;
selecting one of the plurality of springs from the kit based on the desired amount of relative movement restriction determined in the determining step; and
implanting an intervertebral disc prosthesis between the first vertebra and the second vertebra, said intervertebral disc prosthesis including the one of the plurality of springs selected in the selecting step.

23. The method of claim 22, wherein said intervertebral disc prosthesis further includes:
an upper prosthesis component having (i) a first vertebra facing surface, and (ii) a first bearing surface;
a lower prosthesis component having (i) a second vertebra facing surface, and (ii) a second bearing surface; and
an intermediate prosthesis component having (i) a third bearing surface configured to mate with said first bearing surface of said upper prosthesis component, and (ii) a fourth bearing surface configured to mate with said second bearing surface of said lower prosthesis component.

24. The method of claim 23, further comprising attaching the one of the plurality of springs selected in the selecting step to both the upper prosthesis component and the lower prosthesis component.

25. The method of claim 24, further comprising:
attaching the upper prosthesis component to the first vertebra; and
attaching the lower prosthesis component to the second vertebra.

26. The method of claim 25, further comprising positioning the intermediate prosthesis component so that (i) the third bearing surface is in contact with the first bearing surface of the upper prosthesis component, and (ii) the fourth bearing surface is in contact with the second bearing surface of the lower prosthesis component.

27. The method of claim 22, wherein:
said plurality of springs are color coated based upon spring rate,
said first spring possesses a first color, and
said second spring possesses a second color which is different from said first color.

28. The method of claim 22, wherein the one of the plurality of springs selected in the selecting step is made of a nitinol material.

29. A method, comprising:
determining a desired relative movement restriction between a first vertebra and a second vertebra of a patient based on physical condition of the patient;
providing a kit having a plurality of springs;
selecting a first spring and a second spring from the kit based on the desired relative movement restriction determined in the determining step, said first spring possessing a first spring rate, and said second spring possessing a second spring rate that is different from said first spring rate; and
implanting an intervertebral disc prosthesis between the first vertebra and the second vertebra, said intervertebral disc prosthesis including the first spring and the second spring selected in the selecting step.

30. The method of claim 29, wherein said intervertebral disc prosthesis further includes:
an upper prosthesis component having (i) a first vertebra facing surface, and (ii) a first bearing surface;
a lower prosthesis component having (i) a second vertebra facing surface, and (ii) a second bearing surface; and
an intermediate prosthesis component having (i) a third bearing surface configured to mate with said first bearing surface of said upper prosthesis component, and (ii) a fourth bearing surface configured to mate with said second bearing surface of said lower prosthesis component.

31. The method of claim 30, further comprising:
attaching the first spring selected in the selecting step to both the upper prosthesis component and the lower prosthesis component, and
attaching the second spring selected in the selecting step to both the upper prosthesis component and the lower prosthesis component.

32. The method of claim 31, further comprising:
attaching the upper prosthesis component to the first vertebra; and
attaching the lower prosthesis component to the second vertebra.

33. The method of claim 32, further comprising positioning the intermediate prosthesis component so that (i) the third bearing surface is in contact with the first bearing surface of the upper prosthesis component, and (ii) the fourth bearing surface is in contact with the second bearing surface of the lower prosthesis component.

34. The method of claim 29, wherein:
said plurality of springs are color coated based upon spring rate,
said first spring possesses a first color, and
said second spring possesses a second color which is different from said first color.

35. The method of claim 29, wherein both the first spring and the second spring are made of a nitinol material.

36. An intervertebral disc prosthesis kit, comprising:
an upper prosthesis component having (i) a first vertebra facing surface, and (ii) a first bearing surface;
a lower prosthesis component having (i) a second vertebra facing surface, and (ii) a second bearing surface;
an intermediate prosthesis component having (i) a third bearing surface positioned configured to mate with said first bearing surface of said upper prosthesis component, and (ii) a fourth bearing surface configured to mate with said second bearing surface of said lower prosthesis component; and
a plurality of springs including (i) a first spring possessing a first spring rate, and (ii) a second spring possessing a second spring rate that is different from said first spring rate.

37. The method of claim 36, wherein:
said plurality of springs are color coated based upon spring rate,
said first spring possesses a first color, and
said second spring possesses a second color which is different from said first color.

38. The method of claim 36, wherein both the first spring and the second spring are made of a nitinol material.
